(19) United States
(12) Patent Application Publication
(10) Pub. No.: US 2007/0123822 A1
(43) Pub. Date: May 31, 2007

(54) Safety syringe for taking blood

(75) Inventors: Shih-Chun Wang, Chia-Yi (TW); Kiwi Yuan, Taipei (TW); Jia-Ming Chang, Banciao City (TW)

Correspondence Address: BACON & THOMAS, PLLC, 625 SLATERS LANE, FOURTH FLOOR, ALEXANDRIA, VA, 22314, US

(73) Assignee: Biotop Holding Co., Ltd., George Town (KY)

(21) Appl. No.: 11/286,420

(22) Filed: Nov. 25, 2005

(51) Int. Cl. A61M 5/00

(52) U.S. Cl. 604/110; 600/576

(57) ABSTRACT

A safety syringe for taking blood has a hollow barrel having a bottom open end, a top end, a needle hub movably received between the cap and the stop of the barrel and a spring urging against the needle hub to move into the barrel after the cap is rotated to force second sectorial portions of the moving block of the needle block misalign with the first sectorial portions of the stop of the barrel so that the needle hub is received inside the barrel.

### BACKGROUND OF THE INVENTION

1. Field of the Invention

The present invention relates to a syringe, and more particularly to a safety syringe that can safely retract a used needle hub back into a barrel and prevent users from being hurt by the used needle.

2. Description of Related Art

There are two types of conventional syringes used for taking blood, one has a hollow barrel, a plunger and a needle hub, and the other has a short holder, an inner needle and a needle hub. The plunger of the first type is received inside the hollow barrel, and the needle hub is connected to the hollow barrel. The short holder of the second type has an outside surface, a bottom surface and an inner space. The needle hub is mounted on the bottom surface of the outside surface and the inner needle mounted on inner space and connects to the needle hub.

When using the first type of the conventional syringe, a user takes blood into the hollow barrel then inserts the collected blood into a vacuum tube. When using the second type of the conventional syringe, the needle hub is inserted into a vein and a vacutainer is connected to the inner needle inside the short holder. Because of the attraction of the vacutainer and the blood pressure, blood will flow into the vacutainer slowly.

However, the used needles extended outside the hollow barrel or the short holder of the conventional syringes will easily hurt users after taking blood. To keep nurses, doctors or health workers who deal with discarded syringes from being injured or infected by used needles, a safety syringe for taking blood is needed.

To overcome the shortcomings of conventional syringes, the present invention provides a safety syringe for taking blood to mitigate or obviate the aforementioned problem.

### SUMMARY OF THE INVENTION

The primary objective of the present invention is to provide a safety syringe for taking blood. The safety syringe for taking blood in accordance with the present invention comprises a hollow barrel, a needle hub and a spring.

The hollow barrel has a bottom open end in communication with an interior of the barrel so that a tube for containing therein blood is adapted to be received in the hollow barrel via extending through the bottom open end of the hollow barrel. A stop is formed on an inner wall of the barrel and has first cutouts defined in a peripheral edge of the stop. The needle hub has a moving block abutting against the stop and having second cutouts defined in an outer peripheral edge of the moving block to correspond to the first cutouts of the stop of the barrel and a seat connected to the moving block and having a first needle extending into the barrel and a second needle for taking blood. The spring is sandwiched between the cap which is rotatably connected to a free end of the barrel and securely engaged with the moving block and the moving block such that when the cap is rotated, the moving block is driven to rotate and thus the recoil force from the spring will push the moving block back into the barrel after the sectorial portions of the moving block are respectively received in the first cutouts of the barrel so that accidental injury to the persons who deal with discarded material is avoided.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a preferred embodiment of a safety syringe for taking blood in accordance with the present invention;

FIG. 2 is an exploded perspective view of the safety syringe for taking blood of the present invention;

FIG. 3 is an exploded perspective view of the safety syringe for taking blood from a different angle;

FIG. 4 is a cross sectional view showing the assembly of the safety syringe of the present invention;

FIG. 5 is an operational cross-sectioned view showing that the needle hub is rotated inside the barrel;

FIG. 6 is a schematic cross sectional view showing the mutual relationship between the stop and the moving block; and

FIG. 7 is an operational view showing that the needle hub is moved into the barrel by the recoil force from the spring.

### DETAILED DESCRIPTION OF THE PREFERRED EMBODIMENT

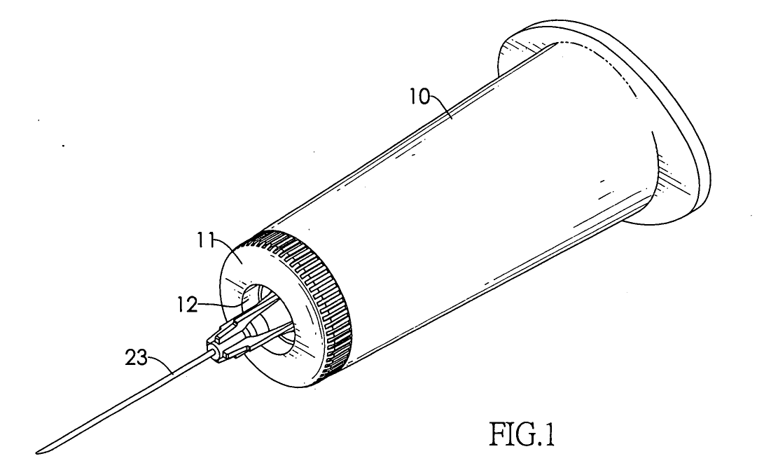

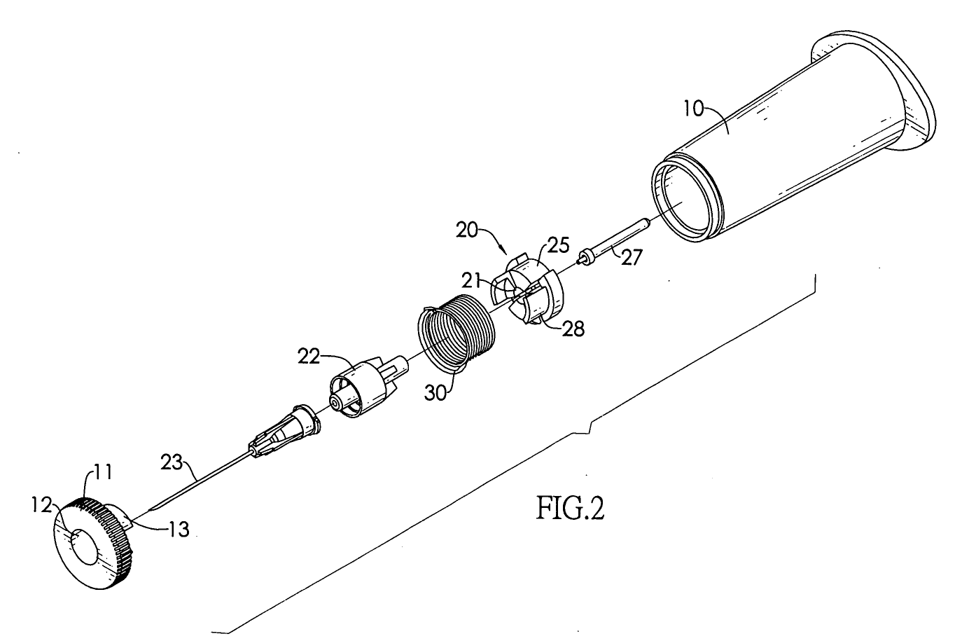

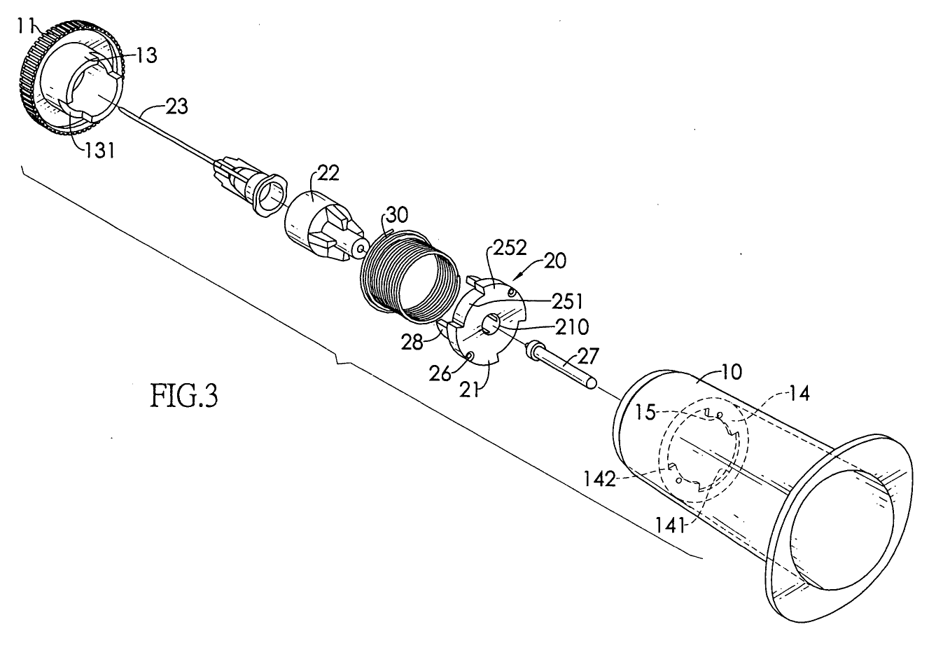

With reference to FIGS. 1, 2 and 3, a safety syringe for taking blood in accordance with the present invention has a hollow barrel (10), a needle hub (20) and a spring (30).

The hollow barrel (10) is cylindrical and has a bottom open end for insertion of a tube and a top end with a cap (11) rotatably mounted to a periphery defining the top open end and having a through hole (12) defined to communicate with an interior of the barrel (10) and a first skirt (13) formed on a side face defining the through hole (12) of the cap (11) to extend toward the interior of the barrel (10) and provided with first notches (131) defined in a side face of the first skirt (13). The barrel (10) has a stop (14) formed on an inner face of the barrel (10) and having first cutouts (141) defined in a periphery of the stop (14) so as to form first sectorial portions (142) on the stop (14). Multiple indents (15) (two are shown) are defined in the stop (14).

The needle hub (20) is composed of a moving block (21) with a passage (210) defined through the moving block (21), a seat (22) with a nose (221) extending from a side of the seat (22) through the passage (210) to firmly connect to a first needle (27) and a second needle (23) extending in a direction opposite to the first needle (27). The moving block (21) further has multiple second cutouts (251) defined in a periphery of the moving block (21) to correspond to the first cutouts (141) of the stop (14) and multiple second sectorial portions (252) formed to correspond to the first sectorial portions (141) of the stop (14). Multiple (two are shown) bosses (26) are formed on a side face of the moving block (21) to correspond to the indents (15) of the stop (14). In addition, the moving block (21) has a second skirt (28) extending from a side of the moving block (21) toward the first skirt (13) and having multiple second notches (281) defined in a periphery of the second skirt (28).

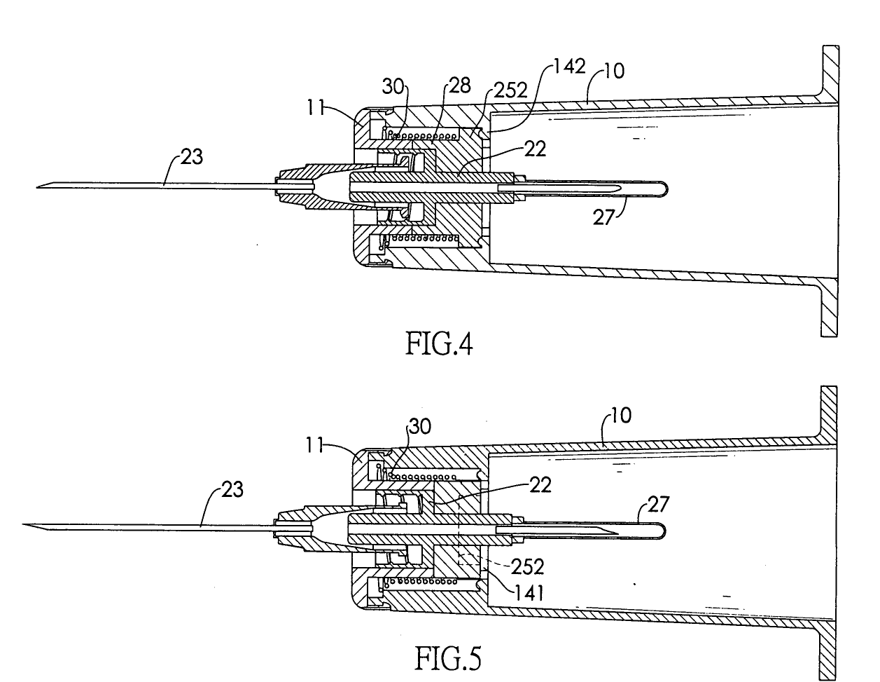

With reference to FIG. 4, when the safety syringe in accordance with the present invention is assembled, the first needle (27) is extended through the passage (210) to firmly engage with the nose (221) of the seat (22) and the second needle (23) is firmly connected to the seat (22) opposite to the first needle (27). The bosses (26) of the moving block (21) are received in the indents (15) of the stop (14) such that the first sectorial portions (142) are aligned with the second sectorial portions (252). Further, the spring (30) is sandwiched between the cap (11) and the moving block (21) after portions of the first skirt (13) are received in the second notches (281) of the second skirt (28). Thereafter, the user is able to use the safety syringe of the present invention to take blood samples.

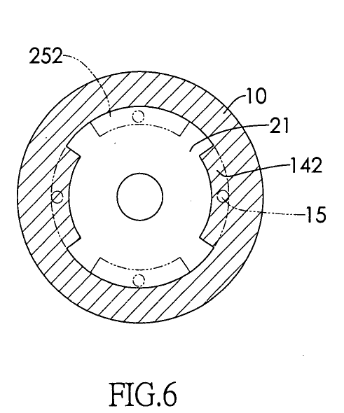

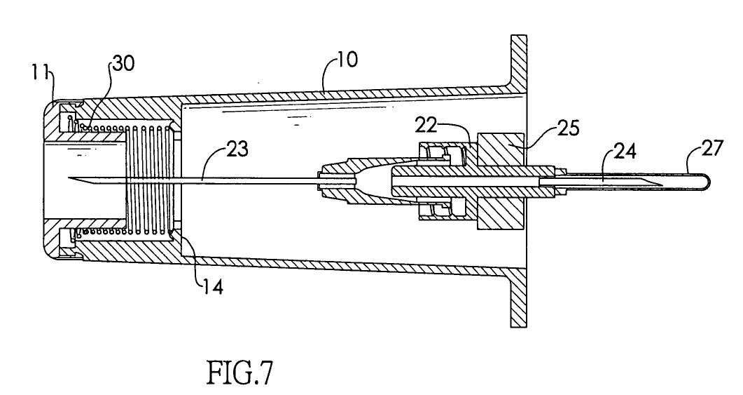

With reference to FIGS. 5 and 6, after the application of the safety syringe of the present invention, the user rotates the cap (11), due to the secure engagement between the cap and the moving block (21), the moving block (21) is also driven to rotate, which misaligns the first sectorial portions (142) to the second sectorial portions (252). That is, when the cap (11) is rotated to a predetermined position, the second sectorial portions (252) are aligned with the first cutouts (141) so that the moving block (21) together with the first needle (27), the seat (22) and the second needle (23) are pushed into the barrel (10) by the spring (30) as shown in FIG. 7. Thereafter, the spring (30) is then sandwiched between the stop (14) and the cap (11).

From the foregoing description, it is noted that the user's safety is protected because all the user needs to do is to rotate the cap (11) and the needle hub (20) will be pushed into the barrel (10) for disposal. Therefore, the user is free from accidental injury by the needle.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A safety syringe for taking blood comprising
a hollow barrel having a bottom open end for receiving therein a tube, a top end and a stop formed on an inner face of the barrel;
a needle hub rotatably connected to the stop of the barrel and having a first needle extending in a first direction toward the barrel and a second needle extending in a second direction opposite to that of the first direction;
a cap rotatably mounted to the top open end of the barrel and detachably connected to the needle hub; and
a spring sandwiched between the needle hub and the cap when the spring is at a first position and sandwiched between the cap and the stop when the spring is at a second position such that after the needle hub is rotated due to the rotation of the cap for predetermined degrees the needle hub together with the first needle and the second needle is pushed by the spring into the barrel for disposal.

2. The safety syringe as claimed in claim 1, wherein the stop includes first cutouts defined in a periphery of the stop and multiple first sectorial portions each formed adjacent to one of the first cutouts,
the needle hub is composed of a moving block configured to have second cutouts corresponding to and aligning with the first cutouts of the stop and second sectorial portions corresponding to and aligning with the first sectorial portions of the stop when the spring is at the first position.

3. The safety syringe as claimed in claim 2, wherein the cap has a first skirt and first notches defined in a side face of the first skirt, the moving block has a second skirt extending from a side face of the moving block and having second notches defined in a side face of the second skirt so that portions of the first skirt are received in the second notches and portions of the second skirt are received in the first notches to allow the moving block to move along with the cap.

4. The safety syringe as claimed in claim 2, wherein the first cutouts and the first sectorial portions of the stop misalign respectively with second cutouts and second sectorial portions of the moving block when the spring is at the second position.

5. The safety syringe as claimed in claim 3, wherein the first cutouts and the first sectorial portions of the stop misalign respectively with second cutouts and second sectorial portions of the moving block when the spring is at the second position.

6. The safety syringe as claimed in claim 1, wherein the cap has a first skirt extending from a side face of the cap and having first notches defined in a side face of the first skirt, the moving block has a second skirt extending from a side face of the moving block to correspond to the first skirt and having second notches defined in a side face of the second skirt such that after first protrusions formed on the first skirt are received in the second notches and second protrusions formed on the second skirt are received in the first notches, engagement between the cap and the moving block is secured.

7. The safety syringe as claimed in claim 2, wherein the cap has a first skirt extending from a side face of the cap and having first notches defined in a side face of the first skirt, the moving block has a second skirt extending from a side face of the moving block to correspond to the first skirt and having second notches defined in a side face of the second skirt such that after first protrusions formed on the first skirt are received in the second notches and second protrusions formed on the second skirt are received in the first notches, engagement between the cap and the moving block is secured.

8. The safety syringe as claimed in claim 3, wherein the cap has a first skirt extending from a side face of the cap and having first notches defined in a side face of the first skirt, the moving block has a second skirt extending from a side face of the moving block to correspond to the first skirt and having second notches defined in a side face of the second skirt such that after first protrusions formed on the first skirt are received in the second notches and second protrusions formed on the second skirt are received in the first notches, engagement between the cap and the moving block is secured.

9. The safety syringe as claimed in claim 4, wherein the cap has a first skirt extending from a side face of the cap and having first notches defined in a side face of the first skirt, the moving block has a second skirt extending from a side face of the moving block to correspond to the first skirt and having second notches defined in a side face of the second skirt such that after first protrusions formed on the first skirt are received in the second notches and second protrusions formed on the second skirt are received in the first notches, engagement between the cap and the moving block is secured.

10. The safety syringe as claimed in claim 4, wherein the cap has a first skirt extending from a side face of the cap and having first notches defined in a side face of the first skirt, the moving block has a second skirt extending from a side face of the moving block to correspond to the first skirt and having second notches defined in a side face of the second skirt such that after first protrusions formed on the first skirt are received in the second notches and second protrusions formed on the second skirt are received in the first notches, engagement between the cap and the moving block is secured.

11. The safety syringe as claimed in claim 2, wherein the stop is provided with indents defined in a side face of the stop and the moving block has bosses formed on to correspond to and be received in the indents when the spring is at the first position and the bosses are moved away from the corresponding indents when the spring is at the second position.

12. The safety syringe as claimed in claim 3, wherein the stop is provided with indents defined in a side face of the stop and the moving block has bosses formed on to correspond to and be received in the indents when the spring is at the first position and the bosses are moved away from the corresponding indents when the spring is at the second position.

13. The safety syringe as claimed in claim 5, wherein the stop is provided with indents defined in a side face of the stop and the moving block has bosses formed on to correspond to and be received in the indents when the spring is at the first position and the bosses are moved away from the corresponding indents when the spring is at the second position.

14. The safety syringe as claimed in claim 6, wherein the stop is provided with indents defined in a side face of the stop and the moving block has bosses formed on to correspond to and be received in the indents when the spring is at the first position and the bosses are moved away from the corresponding indents when the spring is at the second position.

15. The safety syringe as claimed in claim 7, wherein the stop is provided with indents defined in a side face of the stop and the moving block has bosses formed on to correspond to and be received in the indents when the spring is at the first position and the bosses are moved away from the corresponding indents when the spring is at the second position.

16. The safety syringe as claimed in claim 8, wherein the stop is provided with indents defined in a side face of the stop and the moving block has bosses formed on to correspond to and be received in the indents when the spring is at the first position and the bosses are moved away from the corresponding indents when the spring is at the second position.

17. The safety syringe as claimed in claim 9, wherein the stop is provided with indents defined in a side face of the stop and the moving block has bosses formed on to correspond to and be received in the indents when the spring is at the first position and the bosses are moved away from the corresponding indents when the spring is at the second position.

18. The safety syringe as claimed in claim 10, wherein the stop is provided with indents defined in a side face of the stop and the moving block has bosses formed on to correspond to and be received in the indents when the spring is at the first position and the bosses are moved away from the corresponding indents when the spring is at the second position.
